(19) United States
(12) Patent Application Publication
(10) Pub. No.: US 2025/0042747 A1
(43) Pub. Date: Feb. 6, 2025

(54) METHOD FOR PRODUCING PHOSGENE

(71) Applicant: Covestro Deutschland AG, Leverkusen (DE)

(72) Inventors: Andreas Bulan, Langenfeld (DE); Rainer Weber, Odenthal (DE); Alexander Lueken, Kaarst (DE)

(21) Appl. No.: 18/696,188

(22) PCT Filed: Sep. 29, 2022
(86) PCT No.: PCT/EP2022/077162

§ 371 Date: Mar. 27, 2024

(30) Foreign Application Priority Data
Oct. 6, 2021 (EP) 21201336.1

(51) Int. Cl. C01B 32/80; C01B 32/40; C07C 263/10

(52) U.S. Cl. CPC C01B 32/80; C01B 32/40; C07C 263/10

(57) ABSTRACT

A method for producing phosgene from chlorine and carbon monoxide (1), the method being characterised in that carbon monoxide obtained by catalytic decomposition of methanol (4) is provided and this provided carbon monoxide (2) is reacted with chlorine to form phosgene (3). Using this method, low-emission phosgene for the production of isocyanates and polycarbonates can be provided, despite possible fluctuations in the provision of renewable energies. Methanol serves as the source for carbon monoxide, the methanol used for this purpose preferably being provided using regenerative energy from hydrogen and COx, where x=1 or 2.

### CROSS REFERENCE TO RELATED APPLICATIONS

This application is a U.S. national stage application, filed under 35 U.S.C. § 371, of International Application No. PCT/EP2022/077162, which was filed on Sep. 29, 2022, and which claims priority to European Patent Application No. 21201336.1, which was filed on Oct. 6, 2021. The contents of each are hereby incorporated by reference into this specification.

### FIELD

The present invention relates to a process in which utilization products, especially synthesis gas, are obtained from methanol and used for provision as reactant for the chemical production of phosgene, which can be converted in turn, for example chemically, to organic isocyanate or to polycarbonate.

### BACKGROUND

The industrial scale production of di- and polyisocyanates by reacting the corresponding organic amines with phosgene has long been known from the prior art; the reaction can be conducted in the gas or liquid phase and batchwise or continuously (W. Siefken, Liebigs Ann. 562, 75-106 (1949)). There have already been multiple descriptions of processes for producing organic isocyanates from primary amines and phosgene; see, for example, Ullmanns Encyklopadie der technischen Chemie [Ullmann's Encyclopedia of Industrial Chemistry], 4th ed. (1977), volume 13, p. 351 to 353, and G. Wegener et al. Applied Catalysis A: General 221 (2001), p. 303-335, Elsevier Science B.V. There is use here on a global scale both of aromatic isocyanates, for example methylene diphenyl diisocyanate (MMDI—“monomeric MDI”), polymethylene polyphenylene polyisocyanates (i.e. the higher homologs of MMDI, including PMDI, “polymeric MDI”; these are always obtained in industry in a mixture with MMDI components) or tolylene diisocyanate (TDI), and of aliphatic isocyanates, for example hexamethylene diisocyanate (HDI) or isophorone diisocyanate (IPDI).

Polycarbonates are produced on an industrial scale predominantly by reaction of a diphenol or multiple different diphenols and a carbonyl halide by the interfacial process. Polycarbonate production by the interfacial process is already from Schnell, “Chemistry and Physics of Polycarbonates”, Polymer Reviews, Volume 9, Interscience Publishers, New York, London, Sydney 1964, p. 33-70; D. Freitag, U. Grigo, P.R. Müller, N. Nouvertne′, BAYER AG, “Polycarbonates” in Encyclopedia of Polymer Science and Engineering, Volume 11, Second Edition, 1988, p. 651-692. The interfacial process for producing polycarbonate is moreover also described in EP 0 517 044 A2 or EP 520 272 A2.

Polycarbonate is produced by the interfacial process by reacting an initial charge of a disodium salt of a diphenol in aqueous alkaline solution or suspension or of a mixture of two or more different diphenols in aqueous alkaline solution or suspension with a carbonyl halide, especially with phosgene, in the presence of an inert organic solvent or solvent mixture.

The industrial scale production of phosgene which is used in the phosgenation of the corresponding organic amines from CO and chlorine over activated carbon catalysts in a shell-and-tube reactor is likewise known from the prior art (e.g. Ullmann's Encyclopedia of Industrial Chemistry, 5th ed. vol. A 19p 413f., VCH Verlagsgesellschaft mbH, Weinheim, 1991). This involves combining carbon monoxide in a stoichiometric excess with chlorine, and passing them over a fixed bed catalyst. The catalyst used for industrial purposes is activated carbon; the selection of a suitable activated carbon is empirical to date (Mitchell et al.: Selection of carbon catalysts for the industrial manufacture of phosgene; Catal. Sci. Technol., 2012, 2, 2109-2115).

The organic amines needed for isocyanate production are typically produced on an industrial scale by hydrogenation of the corresponding organic nitroaromatics with hydrogen using a catalyst. For example, document EP 1 882 681 A describes a process for adiabatic hydrogenation of nitroaromatics to aromatic amines in the gas phase over fixed catalysts, in which the nitroaromatic to be converted is passed over the catalyst at elevated temperature together with hydrogen, water, and optionally nitrogen, and essentially in the absence of aromatic amine produced from the nitroaromatic.

Chlorine is produced industrially by the conversion of HCl (electrolysis or catalytic conversion) or NaCl (electrolysis). HCl recycling processes are known from the prior art (cf. Ullmann Enzyklopadie der technischen Chemie, Weinheim, 4th edition 1975, vol. 9, pp. 357 ff. or Winnacker-Küchler: Chemische Technik, Prozesse und Produkte [Chemical Technology, Processes and Products], Wiley-VCH Verlag, 5th Edition 2005, Vol. 3, pp. 512 ff.): these particularly include the electrolysis of hydrochloric acid and the oxidation of hydrogen chloride. The process developed by Deacon in 1868 for catalytic hydrogen chloride oxidation with oxygen in an exothermic equilibrium reaction was at the genesis of industrial chlorine chemistry:

4HCl+O₂ [symbol] 2Cl₂+2H₂O

Although the electrolysis of NaCl (chlor-alkali process) has pushed the Deacon process into a secondary role, it is nevertheless employed industrially. Virtually all chlorine was produced by electrolysis of aqueous sodium chloride solutions (Ullmann Encyclopedia of Industrial Chemistry, seventh release, 2006).

Hydrogen and carbon monoxide are nowadays generated mainly by the steam reforming process. This involves converting hydrocarbons by means of steam using a heterogeneous catalyst to the hydrogen and carbon monoxide products. The starting material used here is mainly natural gas, although it is also possible to use metallurgical gases, residual materials (for example plastic packaging waste, mattresses or other polymeric residual materials) or naphtha.

The endothermic reaction (process temperature: 800-900° C.), which is typically conducted in a shell-and-tube reactor filled with a nickel catalyst, is composed of the following processes/reactions:

CH₄+H₂O [symbol] CO+3H₂

CO+H₂O [symbol] CO₂+H₂

The composition of the product gas depends significantly on the process parameters. The most important parameters here are pressure, reforming gas temperature, and the ratio between steam and hydrocarbons.

With regard to the use of natural gas, it should be added that natural gas has to be purified before it can be used in the process. It is necessary here to not just to separate high boilers, but also to remove possible catalyst poisons, from the feed gas. It is essential here to remove sulfur, which is a particularly potent catalyst poison. As well as natural gas, it is also possible to use higher hydrocarbons. However, pre-reforming steps are needed for the purpose.

In the recovery of synthesis gas, energy coupling can be achieved by the combination of multiple processes. Here, the endothermic steam reforming process

CH₄+H₂O [symbol] CO+3H₂

is coupled to exothermic partial oxidation (partial combustion of hydrocarbons, which results in a H₂-rich synthesis gas; see ULLMANN'S Encyclopedia of Industrial Chemistry: “Gas production”, 2016), such that the steam reformer is heated with the hot exit gas from the partial oxidation. Such a combination of the processes mentioned was described, for example, in U.S. Pat. No. 6,730,285B2.

Depending on the end use, the synthesis gas obtained is subjected to further treatment steps. For example, the water-gas shift reaction is utilized industrially in order to increase the hydrogen content in existing CO/H₂ mixtures. This involves reacting CO with H₂O over a heterogeneous catalyst:

CO+H₂O [symbol] CO₂+H₂

This process can be utilized under higher (350-400° C.) or else milder conditions (200-250° C.).

A further process for producing a CO-containing synthesis gas is electrochemical CO₂ reduction. The CO₂ electrolysis here may be, for example, a high-temperature electrolysis which is operated at a temperature of more than 600° C., possibly with the addition of water for production of synthesis gas. High-temperature electrolyses are known in principle and available on the market, e.g. from Haldor Topsoe, eCOs™. During high-temperature electrolysis, oxygen is also produced at the anode.

In the case of low-temperature electrolysis, CO₂ is in particular converted to carbon monoxide and possibly hydrogen at a gas diffusion electrode. At the same time, O₂ or, in some cases, alternatively chlorine as well can be generated at the anode. The process is described by way of example in T. Haas et al., Nature Catalysis 2018, 1, 32-39.

According to the known principles, an MEA (membrane electrode assembly) concept can also be used in low-temperature electrolysis. In this case, a catalyst is applied to the membrane. An upstream gas diffusion layer regulates gas and liquid transport. This can be effected on both the anode and the cathode side. It is also possible to bring a gas diffusion electrode into direct contact with the membrane.

A further method of producing a CO-containing synthesis gas is the reverse water-gas shift reaction (RWGS).

Further purification of the synthesis gas constituents depends on the subsequent production step. For example, for the synthesis of ammonia, all CO₂ components must be removed, which is typically effected by scrubbing processes. Traces of CO are removed here via methanation or N2 scrubbing. In addition, as required, it is also possible to use pressure swing adsorptions for further purification.

Methanol is nowadays produced on an industrial scale almost exclusively by what is called the low-pressure process, which works within pressure ranges between 50 and 120 bar. Compared to the historical high-pressure processes that operate at 250-400 bar, it was thus possible to drastically lower production costs and capital costs. The reduction in process pressure was ultimately enabled by the use of Cu/ZnO catalysts rather than ZnO/Cr₂O₃ catalysts. This switch was brought about in particular by advances in the process regime (including reduction of possible catalyst poisons in the feed). The particular benefit of the Cu/ZnO catalyst here is that the synthesis can be conducted at significantly lower temperatures (200-300° C.), which ultimately requires lower system pressures.

For the methanol synthesis, carbon oxides are converted together with hydrogen (synthesis gas). This process is exothermic and can be described by the following reaction equations:

CO+2H₂ [symbol] CH₃OH

CO₂+3H₂O [symbol] CH₃OH+H₂O

Both reactions are coupled by the likewise exothermic water-gas shift reaction, which can be described as follows:

CO+H₂O [symbol] CO₂+H₂

What is typically used for the methanol synthesis is synthesis gas consisting mainly of CO and H₂, the composition of which has a major influence on the optimal utilization. According to D1, the composition can be described via the stoichiometric ratio (SN):

$$SN = \frac{{n}_{{H}_{2}} - {n}_{{CO}_{2}}}{{n}_{CO} + {n}_{{CO}_{2}}}$$

When SN=2, the reactants are present in a stoichiometric ratio according to the above reaction equations. In real terms, however, slightly higher values for SN (2.01-2.1) are used, which is achieved by a higher hydrogen content in the synthesis gas (Dittmeyer et al. “Chemische Technik” [Chemical Technology], volume 4, 5th edition). In spite of this optimized composition, however, complete conversion is impossible from a thermodynamic point of view. Recycling of the unconverted reactants is therefore absolutely necessary.

As a result of the processes that occur in the reactor, the product exiting from the reactor has to be purified further. For example, significant amounts of water, but also small amounts of further by-products such as dimethyl ether, methyl formate or higher alcohols, are present. The product mixture is typically separated here by distillation.

With regard to technological aspects, methanol synthesis is considered to be very substantially optimized. Nevertheless, further optimization can be achieved, for example by improving the exchange of energy between the individual process stages. For example, in the recovery of synthesis gas, energy coupling can be achieved by the combination of multiple processes. Here, the endothermic steam reforming process

CH₄+H₂O [symbol] CO+3H₂

is coupled to endothermic partial oxidation (partial combustion of hydrocarbons, which results in a H₂-rich synthesis gas; see U.S. Pat. No. 6,340,437 B1), such that the steam reformer is heated with the hot exit gas from the partial oxidation. Such a combination of the processes mentioned was described, for example, in U.S. Pat. No. 6,730,285 B2.

While methanol is produced in virtually all industrially operated plants by the reaction of CO and H₂, the use of CO₂ (see above) as raw material (see, for example, ChemCatChem 2019, 11, 4238-4246) is being pursued ever more intensively. ChemCatChem 2019, 11, 4238-4246 describes the equilibrium yield at 230° C. and 50 bar as 30%.

The production of synthesis gas by the degradation of methanol is a known and commercial process. The process can be regarded as a reverse reaction of the above-described industrial methanol production, since the reaction proceeds via the same chemical equilibrium. However, it is known (Topics in Catalysis vol. 22, nos. 3-4, April 2003) that, in spite of the same net reaction equation, the mechanism of the reverse reaction differs from methanol synthesis. In the absence of water, methyl formate is formed here at first with release of hydrogen and then breaks down to CO and H₂:

2CH₃OH→CH₃OCHO+2H₂

CH₃OCHO→2CO+2H₂

In the presence of water, the reaction forms the basis for the commonly known methanol reformer. Target products here, analogously to the water-gas shift reaction, are CO₂ and H₂:

CH₃OH+H₂O [symbol] CO₂+3H₂

U.S. Pat. No. 6,583,084 describes the production of hydrogen by the degradation of methanol in the gas phase. Cu-based catalysts are used.

EP0409517 describes a process for producing synthesis gas from methanol. Catalysts used here are Cr- and Zn-based materials. The catalyst may be doped here with various materials. The degradation takes place at a temperature between 270° C. and 400° C., and a pressure of 20 kg cm⁻² (corresponding to about 20 bar). The reaction is conducted in a shell-and-tube reactor.

US 20050108941 A describes a process for producing hydrogen or synthesis gas by conversion of methanol in a suspension. This involves using a catalyst consisting of copper, zinc oxide, aluminum and chromium.

U.S. Pat. No. 6,699,457 describes a process for producing hydrogen or synthesis gas by conversion of methanol in a suspension or in the gas phase. The active catalyst component used here is a group VIII transition metal. The process is conducted here, for example, at a temperature below 400° C., where the pressure in the system is adjusted such that water and the hydrocarbon used (“oxygenated hydrocarbon”) are in the form of liquids.

It can be summarized that, as well as the choice of catalyst, the presence of water also has a significant influence on the selectivity of the process. The presence of water can, for example, lower the proportion of CO in the synthesis gas.

A further process for producing hydrogen is the electrochemical conversion of water. Water is split here into hydrogen and oxygen using electricity. Known processes are proton exchange electrolysis (PEM), which is described, for example, in EP 3489394 A. A further process is alkaline water electrolysis. The two processes differ in particular by the process parameters, for example temperature, pressure and pH. However, the two processes lead to the hydrogen and oxygen target products.

### SUMMARY

The problem addressed by the present invention was that of providing a process for producing phosgene, with the aid of which, in spite of possible fluctuations in the provision of renewable energies, a low-emission phosgene can be provided for the production of isocyanates and polycarbonates.

The invention firstly provides a process for producing phosgene from chlorine and carbon monoxide, in which carbon monoxide obtained by catalytic degradation of methanol is provided and the carbon monoxide provided is reacted with chlorine to give phosgene.

### BRIEF DESCRIPTION OF DRAWINGS

The process and preferred embodiments thereof are illustrated by way of example in the figures FIG. 1 and FIG. 2, without restricting the subject matter of the invention thereto. Arrows in the figure symbolize the flow of substances, energy or heat between process steps/apparatus parts in which the corresponding process steps proceed. Dashed lines in the figures denote parts of preferred embodiments of individual above-described features of the process. Dashed-and-dotted frames illustrate the region of origin of the raw materials provided for the process of the invention, methanol and carbon monoxide here. The following reference numerals are used in the figures:
1 Production of phosgene from chlorine and carbon monoxide
2 Carbon monoxide provided, which is a process product of a catalytic degradation of methanol (4)
3 Phosgene
4 Catalytic degradation of methanol
5 Methanol provided, which is a process product of a reaction 6 selected from an electrochemical reaction, a homogeneously catalyzed reaction or a heterogeneously catalyzed reaction
6 Production of methanol by reaction of a gas stream with COₓ in which x=1 or 2 and with optionally hydrogen (preferably of at least CO₂ and hydrogen), where the methanol produced is a process product of a reaction selected from an electrochemical reaction, a homogeneously catalyzed reaction or a heterogeneously catalyzed reaction
7 Electrical power from renewable energy
8 Electrolysis of water
9 Isocyanate production and/or polycarbonate production
10 Region of origin of the carbon monoxide 2 provided
11 Region of origin of the methanol 5 provided
12 Storage of methanol which is a process product of the production 6

FIG. 1 shows an embodiment of a process of the invention in which carbon monoxide 2, which is a process product of a catalytic degradation of methanol 4, is provided, for example, by withdrawal of corresponding methanol from a storage tank or from a conduit in fluid connection with the catalytic degradation of methanol 4. This provided carbon monoxide 2 is directed into a production of phosgene 1, where it is reacted with chlorine (Cl₂) to give phosgene 3, and conducted out of the production of phosgene 1, preferably followed by supply of phosgene 3 into an isocyanate production 9. The methanol 5 provided in FIG. 1 is preferably a process product of a production of methanol 6 by reaction of hydrogen with at least CO₂, where the methanol produced is a process product of a reaction selected from an electrochemical reaction, a homogeneously catalyzed reaction or a heterogeneously catalyzed reaction. It is particularly preferable here in turn when hydrogen that has been by electrolysis 8 of water with use of electrical power from renewable energy 7 is used. The region 10 of production of said carbon monoxide 2 provided marks corresponding process steps or plant components that are implemented for the provision by suppliers or the phosgene producer itself. The region 11 of production of the methanol 5 provided marks corresponding process steps or plant components that can be implemented for the provision by a supplier of the carbon monoxide 2 provided, by a further supplier of methanol 5 provided or by the phosgene producer itself.

FIG. 2 shows an embodiment of a process of the invention in which carbon monoxide 2, which is a process product of a catalytic degradation of methanol 4, by withdrawal of corresponding methanol from a storage tank 12 which is in fluid connection with the catalytic degradation of methanol 4, is directed into a production of phosgene 1, where it is reacted with chlorine (Cl₂) to give phosgene 3, and conducted out of the production of phosgene 1, preferably followed by supply of phosgene 3 to an isocyanate production 9. The degradation of methanol 4 releases hydrogen gas which is fed to the production of methanol 6. The production of methanol 6 takes place by reaction of hydrogen with at least CO₂, where the methanol produced is a process product of a reaction selected from an electrochemical reaction, a homogeneously catalyzed reaction or a heterogeneously catalyzed reaction. If the amount of hydrogen gas obtained from the degradation of methanol 4 is insufficient for the production of methanol 6, it is particularly preferable when additional hydrogen is utilized, which is produced by electrolysis 8 of water using electrical power from renewable energy 7.

### DETAILED DESCRIPTION

It is sufficient for the provision of the carbon monoxide in the context of the invention when the carbon monoxide is a process product of a catalytic degradation of methanol. This means that, when performing the process of the invention, it is sufficient for the execution of the step of providing the carbon monoxide even to merely draw said carbon monoxide from a storage vessel or from a feed conduit in the context of a delivery as raw material. In this case, the phosgene producer, as implementer of the process of the invention, does not itself execute the catalytic degradation of methanol for production of the carbon monoxide, but merely ensures that the carbon monoxide provided has been produced by a supplier by catalytic degradation of methanol and is the process product thereof.

It is likewise possible in accordance with the invention, in one embodiment, when the carbon monoxide is provided in that the aforementioned catalytic degradation of methanol is executed by the phosgene producer itself as integral steps of a process of the invention for production of phosgene, and the carbon monoxide obtained is sent to phosgene production.

These means of provision, i.e. supply or in-house production by the phosgene producer, are likewise also applicable to the embodiments of the provision of carbon monoxide that are mentioned below.

In the process of the invention, the carbon monoxide provided for use in phosgene production is produced from methanol. In a preferred embodiment of the invention, methanol is provided for the purpose beforehand, which is the process product of an electrochemical reaction, a homogeneously catalyzed reaction or a heterogeneously catalyzed reaction.

For the provision of methanol, it is accordingly sufficient when the methanol is a process product of at least one of the aforementioned reactions. This means that it is sufficient for the execution of the step of providing the methanol even to merely draw said methanol from a storage vessel or from a feed conduit in the context of a delivery as raw material. In this case, the phosgene producer, as implementer of the process of the invention, does not itself execute the aforementioned reaction for production of the methanol, but merely ensures that the methanol provided for the catalytic degradation of methanol for provision of carbon monoxide has been produced by employment of at least one of these said methods and hence is the process product thereof.

It is likewise possible in accordance with the invention when the methanol is provided in that at least one of the aforementioned reactions for production of methanol is executed by the phosgene producer as integral steps in a process of the invention for production of phosgene, and the methanol obtained, optionally after intermediate storage in a storage vessel, is sent to the catalytic degradation of methanol with provision of carbon monoxide.

The means of provision, i.e. supply or in-house production of the specific methanol by the phosgene producer, are likewise also applicable to the embodiments of the provision of methanol that are mentioned below.

Particular preference is given to an embodiment in which the methanol to be provided for the process of the invention has been produced using renewable energy, preferably at a geographic site with good availability of renewable energy, in order subsequently to provide this methanol for the process of the invention either by delivery by transport in containers or by a methanol stream in a continuous process. For the provision in a continuous process, methanol production is preferably in fluid connection with methanol degradation, for example via a pipeline.

“Renewable energy” is understood by those skilled in the art to mean energy from an inexhaustible energy source, for example wind energy, hydro energy, bio energy (e.g. conversion of biogas or biomass to power) or solar energy. Suitable renewable energy is therefore most preferably either wind power, solar energy, hydro power or mixtures thereof.

If renewable energy is used as energy source in the context of the present invention, especially for the provision of electrical power, it can then be advantageous for the assurance of execution of the process of the invention in a period of shortage of renewable energy to store renewable energy at a time of availability of renewable energy, for example in the form of electrical power in an accumulator, in order to bridge periods of shortage.

According to the invention, an “electrochemical reaction” takes place by application of electrical current in the reaction medium (e.g. via at least one electrode immersed into the reaction medium) in the presence of at least one reactant (e.g. carbon dioxide or methanol).

According to the invention, a “catalyzed reaction” or “catalytic reaction” takes place using a catalyst that catalyzes product formation from at least one reactant (e.g. carbon dioxide or methanol). According to the invention, a “homogeneously catalyzed reaction” takes place using a homogeneous catalyst, and a “heterogeneous catalyzed reaction” using a heterogeneous catalyst.

Preferably in accordance with the invention, the methanol provided for the degradation is produced from a gas stream comprising at least COₓ with x=1 or 2, with or without hydrogen gas.

In a particularly preferred embodiment of the process, the methanol provided for the degradation is produced from a carbon monoxide- and hydrogen-containing gas stream which is obtained by a method or a combination of methods from the group of steam reforming of methane, partial oxidation of hydrocarbons, gasification of biomass, electrolysis, reverse water-gas shift. More preferably, at least one method is an electrochemical reaction, a homogeneously catalyzed reaction or a heterogeneously catalyzed reaction.

It has further been found to be preferable when, in one embodiment of the process, methanol is obtained by the conversion of CO₂ with or without hydrogen and is provided for the catalytic degradation. It is particularly preferred in turn when the reaction is an electrochemical reaction, a homogeneously catalyzed reaction or a heterogeneously catalyzed reaction. The CO₂ used for the purpose, in a further embodiment, additionally originates from a further CO₂ source, for example the CO₂ emitted in the provision of thermal energy or the CO₂ from an external CO₂ source, for example an industrial offgas.

A preferred CO₂ source is at least one external CO₂ source that contributes CO₂ which is not emitted by the process of the invention. An external CO₂ source would be, for example, the CO₂ which in cement production, is obtained in H₂ production for ammonia synthesis, is obtained in fermentation, forms in the offgas on combustion of fuels (e.g. waste combustion), or CO₂ which is obtained from air. The CO₂ from an external CO₂ source, in a preferred embodiment of the process of the invention, will be effected by absorption of a CO₂ content from (i) process gases or offgases which is selected from at least one process selected from cement production, fermentation, H₂ production, incineration, and/or (ii) from air by introduction into alkali metal hydroxide solution, for example potassium hydroxide solution. This results in the formation of potassium hydrogen carbonate, which can then be thermally decomposed back to CO₂ and potassium hydroxide. The CO₂ released is then fed to the reformer process of the invention for synthesis of carbon monoxide. For provision of the thermal energy needed for the release of CO₂, it is possible, for example, to use the thermal energy generated from said reformer process.

The methanol used for the degradation, in a particularly preferred embodiment of the process, is produced from carbon dioxide, especially (i) by a catalytic conversion of a synthesis gas containing more than 50% by volume of a mixture of carbon dioxide and hydrogen, (ii) by fermentation or (iii) by electrochemical conversion of carbon dioxide. Each synthesis process (i) to (iii) is supplied here with a CO₂-containing stream of matter as feed. In the case of fermentation (ii) and especially of catalytic conversion (i), the feed additionally contains hydrogen. More preferably, the hydrogen required for steps (i) or (ii) is produced by at least one process selected from the electrolysis of water, partial oxidation or biomass gasification, or mixtures thereof. It is very particularly preferable here in turn when electrical power from renewable energy is used for the electrolysis of water, especially selected from solar energy, wind power, hydro power or mixtures thereof. This power from renewable energy may also be taken from a storage medium, for example an accumulator.

Water electrolysis can be conducted with prior art plants. Industrial plants for alkaline water electrolysis as well as for polymer electrolyte-based electrolysis, what is known as PEM electrolysis, are known and commercially available. The principles of water electrolysis are described by way of example in chapter 6.3.4 in Volkmar M. Schmidt in “Elektrochemische Verfahrenstechnik” [Electrochemical Process Technology](2003 Wiley-VCH-Verlag; ISBN 3-527-29958-0).

In a further execution, carbon monoxide and/or water is added as a secondary component to the synthesis gas for the catalytic conversion. Further constituents may be inert gases.

For the electrochemical conversion (iii), a gas stream containing more than 50% by volume of a mixture of carbon dioxide and water vapor is preferred.

In all aforementioned processes (i) to (iii), a methanol-containing crude product is obtained, which is preferably subjected to a processing operation. In the workup, CO₂, CO and H₂ are first removed. This fraction is preferably recycled into the methanol synthesis. Most preferably, a portion of the gas is discharged (purge). After the removal, the remaining mixture is subjected to a distillation or rectification. The target product obtained here is methanol, which is preferably directed from the workup into a methanol reservoir. Further components removed from the distillation may include dimethyl ether and ethanol. A further fraction obtained in the synthesis is water, which is sent either to disposal or preferably to the abovementioned water electrolysis.

In the catalytic degradation step of the process of the invention, methanol provided (for example taken from a storage vessel for provision or produced beforehand by at least one process (i) to (iii) in a continuous process), in a preferred embodiment, is broken down by contacting gaseous methanol with a catalyst and catalytically to form carbon monoxide and optionally additionally hydrogen gas. If the methanol is not in gaseous form for this purpose (for example in the case of withdrawal from a storage vessel), it is subjected to evaporation beforehand. The energy input needed for the evaporation can be generated by utilization of fossil fuels, for example natural gas, as energy source. The process of the invention, for the establishment of the temperature necessary for evaporation, is more preferably supplied with thermal energy which is provided at least by a method selected from (i) the combustion of fuel containing hydrogen (gaseous H₂) produced by means of renewable energy, (ii) the combustion of fuel containing methane from a biological source, or (iii) the conversion of electrical energy generated from renewable energy to heat. It is particularly preferable here in turn when the renewable energy used is either wind power, solar energy, hydro power or mixtures thereof.

Preferably, for catalytic degradation, gaseous methanol is passed over a catalyst, the active component of which comprises at least one transition metal species. A transition metal species is understood by the person skilled in the art to mean all d-block elements and chemical compounds thereof. More preferably, the catalyst is a transition metal species applied to a support. Very particular preference is given to at least one transition metal species selected from group VII, VIII or XI of the Periodic Table of the Elements, preferably applied in turn to a support.

The catalytic degradation of methanol is preferably conducted at a temperature below 500° C., more preferably below 400° C. If an energy input is necessary for the catalytic degradation of methanol, this can be brought about by utilization of fossil fuels, for example natural gas, as energy source. The process of the invention, for the establishment of the temperature necessary for catalytic cracking of the methanol, is more preferably supplied with thermal energy which is provided at least by a method selected from (i) the combustion of fuel containing hydrogen (gaseous H₂) produced by means of renewable energy, (ii) the combustion of fuel containing methane from a biological source, or (iii) the conversion of electrical energy generated from renewable energy to heat. It is particularly preferable here in turn when the renewable energy used is either wind power, solar energy, hydro power or mixtures thereof.

The catalytic degradation of methanol is preferably conducted at a temperature of at least 200° C. It is preferable in turn when, for the catalytic degradation of methanol, the temperature is set at 200 to below 500° C., more preferably at 200 to below 400° C.

The catalytic degradation of methanol is preferably conducted at an absolute pressure of below 50 bar, more preferably of below 40 bar.

The catalytic degradation step is preferably conducted at a temperature below 500° C. and an absolute pressure below 50 bar. More preferably, the catalytic degradation step is conducted at a temperature below 400° C. and an absolute pressure below 40 bar. It is very particularly preferable in turn when the temperature in each case does not go below the lower temperature limit of 200° C.

The product gas obtained from the catalytic degradation contains, as well as carbon monoxide, often additionally methanol, carbon dioxide, hydrogen and water, and possibly by-products that may come from the group of the ethers (dimethyl ether), alcohols (ethanol), aldehydes or esters. The product gas obtained from the catalytic degradation, before reaction with chlorine to give phosgene, in a preferred, additional process step, is preferably subjected to a purification step in which methanol, carbon dioxide, hydrogen and water, and possibly by-products, are separated from the carbon monoxide.

The product gas from the catalytic degradation is consequently preferably purified and fed to a CO₂ removal unit for the purpose of removal of carbon dioxide, in which the CO₂ is removed.

The CO₂ removal unit and hence the removal of CO₂ can be executed as an “amine scrubbing”, where the carbon monoxide-containing process gas from the reformer process here undergoes, in particular, the fundamentally known scrubbing of the gas mixture by the principle of chemisorption with amines, such as monoethanolamine (MEA), diethanolamine (DEA), methyldiethanolamine (MDEA) or diglycolamine (DGA), which achieves a high purity of the purified gas mixture in an absorption column.

In an embodiment of the process which is preferred in accordance with the invention, water is separated from the carbon monoxide-containing product gas.

In a further preferred embodiment of the process, carbon dioxide is separated off after water has been separated from the carbon monoxide-containing product gas beforehand. For this purpose, the carbon monoxide-containing product gas from the catalytic degradation is first fed to a water removal unit, where the water is separated off, and the carbon monoxide-containing, dry product gas obtained after the separation of water is fed to a CO₂ removal unit in which the CO₂ is separated off. In the water removal unit, the water is separated off, for example, by cooling the carbon monoxide-containing product gas removed from the catalytic degradation and separating off the water, for example as condensate.

A variant of the process which is preferred in accordance with the invention is a process in which the carbon monoxide-containing product gas (preferably having been freed of water and of CO₂ beforehand) is introduced into an H₂—CO separation unit in which hydrogen is separated off. This forms at least one gas stream, wherein the gas at 25° C. and 1013 mbar contains at least 95% by volume of carbon monoxide, more preferably at least 99% by volume of carbon monoxide. Said introduced product gas is preferably first separated into two gas streams in the H₂—CO separation unit. This gives rise to a gas in the form of a gas stream containing at least 95% by volume (preferably at least 99% by weight) of carbon monoxide, a further gas in the form of a gas stream having hydrogen as its major constituent and carbon monoxide inter alia. The further gas is also referred to as residual gas from the H₂—CO separation or, if there is no residual gas treatment, as end gas. A H₂—CO separation unit that works by this principle of separation is called the coldbox. The hydrogen-containing residual gas from the H₂—CO separation can be introduced back into the production of methanol.

Methanol as unreacted reactant and by-products, in a preferred embodiment of the process, are removed from the product gas from the catalytic degradation. Reactants and by-products are preferably fed into the preceding process step or further processing steps. Particular preference is given to purification by adsorption or by a thermal separation process, for example distillation or rectification. More preferably, the reactant and by-products are first separated, and the reactants are fed to the degradation of methanol. Most preferably, the hydrogen obtained as the product, as described above, is fed to a methanol synthesis or to a fermentation process.

In a preferred embodiment of the process, hydrogen gas is removed as a constituent of the product gas obtained in the catalytic degradation and reused for the provision of methanol. A preferred embodiment of the process is therefore characterized in that
(i) methanol provided which is fed to the catalytic degradation of methanol is the process product of a reaction of CO₂ with hydrogen gas, and
(ii) the catalytic degradation of the methanol already provided affords not only carbon monoxide but additionally hydrogen gas,
   with the proviso that hydrogen gas formed in the catalytic degradation is fed to the reaction of CO₂ with hydrogen gas to give methanol.

The carbon monoxide present as a result of catalytic degradation of methanol and optionally as a result of purification is converted in a next process step, preferably over a catalyst, together with chlorine to give phosgene. More preferably, the catalyst is activated carbon.

The phosgene obtained is preferably fed to an additional step in the form of a reaction with at least one organic amine or at least one dihydroxyaryl compound and converted chemically. More preferably, the products obtained are subjected to a purification and workup.

In a reaction of phosgene preferred in accordance with the invention for production of an organic isocyanate, preference is generally given to those production processes of the invention in which the organic isocyanate obtained contains at least two isocyanate groups. For this purpose, the reactant used in the synthesis is in turn preferably organic amine having at least two amino groups.

Organic amines used with very particular preference are selected from tolylenediamine (TDA), methylenedi(phenylamine) (MDA) (preferably selected in turn from diphenylmethane-2,2′-diamine, diphenylmethane-2,4′-diamine, diphenylmethane-4,4′-diamine or mixtures thereof), hexamethylenediamine, isophoronediamine, 1,3-bis(aminomethyl)benzene, cyclohexyldiamine or mixtures thereof, where TDA, MDA or mixtures thereof are very particularly preferred organic amines.

More preferably, the organic isocyanate obtained in the reaction contains at least two isocyanate groups and has a molar mass of not more than 1000 g/mol, especially of not more than 800 g/mol.

Organic isocyanates obtained with very particular preference are selected from tolylene diisocyanate (TDI), methylene di(phenyl isocyanate) (MDI) (preferably selected in turn from diphenylmethane 2,2′-diisocyanate, diphenylmethane 2,4′-diisocyanate, diphenylmethane 4,4′-diisocyanate or mixtures thereof), hexamethylene diisocyanate (HDI), isophorone diisocyanate (IPDI), 1,3-bis(isocyanatomethyl)benzene (XDI), cyclohexyl diisocyanate (CHDI) or mixtures thereof, where TDI, MDI or mixtures thereof are very particularly preferred organic isocyanates.

The reaction with organic amines more preferably affords a mixture containing at least one compound selected from tolylene diisocyanate, methylene diphenyl isocyanate (preferably selected from diphenylmethane 2,2′-diisocyanate, diphenylmethane 2,4′-diisocyanate, diphenylmethane 4,4′-diisocyanate or mixtures thereof), hexamethylene diisocyanate, isophorone diisocyanate, 1,3-bis(isocyanatomethyl)benzene, or cyclohexyl diisocyanate. Most preferably, after the reaction, for a purification and workup of said compound, at least the step of distillation is employed.

In an exceptionally preferred embodiment of the invention, the organic amine reacted with phosgene to give the corresponding isocyanate is methylenedi(phenylamine) (MDA) (preferably selected in turn from diphenylmethane-2,2′-diamine, diphenylmethane-2,4′-diamine, diphenylmethane-4,4′-diamine or mixtures thereof). The MDA in the context of this embodiment is more preferably produced at least by the following steps:
provision of methanol which is a process product of a conversion of a carbon monoxide- and hydrogen-containing gas stream which has been obtained by a method or a combination of methods from the group of steam reforming of methane, partial oxidation of hydrocarbons, gasification of biomass, electrolysis, reverse water-gas shift;
reaction of the previously provided methanol with oxygen gas to give formaldehyde;
conversion of aniline and said formaldehyde to MDA.

For this embodiment of MDA production, the methanol, like the methanol for the catalytic degradation, is provided according to at least one of the above-described methods. The embodiments described above for this purpose are applicable mutatis mutandis.

The production of formaldehyde from methanol using oxygen gas is a preparation route known to the person skilled in the art, the execution of which is described, for example, in chapter 4 from “Formaldehyde” in: Ullmann's Encyclopedia of Industrial Chemistry, Wiley-VCH Verlag GmbH & Co. KGaA, Weinheim, 2012, p. 735-768, DOI: 10.1002/14356007, which is expressly fully incorporated here by reference. The oxygen gas used for the conversion preferably comes in turn from electrolysis of water using electrical energy produced from renewable energy, and/or from fractionation of air.

The hydrogen-containing residual gas from an abovementioned H₂—CO separation from CO purification and/or the hydrogen gas from the aforementioned water electrolysis can be used in a hydrogenation of nitrobenzene for provision of the aniline required for MDA production. The performance of a hydrogenation of organic nitro compounds with hydrogen gas is very familiar to the person skilled in the art. Hydrogenation reactions that are correspondingly employable and usable for the purpose are known by the person skilled in the art, for example, from the following publications: Gerald Booth (2007), Ullmanns Enzyklopadie der Industriechemie, Weinheim, Wiley VCH, doi:10.1002/14356007.a17_411, which is expressly fully incorporated here by reference.

In a further embodiment of the process of the invention, a polycarbonate is produced by reacting the phosgene produced in accordance with the invention with at least one dihydroxyaryl compound. This involves dissolving the dihydroxyalkyl compound in an aqueous solution of alkali metal salt. The phosgene is introduced into an organic phase. The phosgene is reacted in a known manner with at least one dihydroxyaryl compound, carbonic acid derivatives, optionally chain terminators and optionally branching agents, and the polyestercarbonate is produced by replacing a portion of the carbonic acid derivatives with aromatic dicarboxylic acids or derivatives of the dicarboxylic acids, and specifically with aromatic dicarboxylic ester structural units according to the carbonate structural units to be replaced in the aromatic polycarbonates.

Dihydroxyaryl compounds suitable for the production of polycarbonate, for the reaction with phosgene, are those of the formula (1):

HO—Z—OH (1)

in which
Z is an aromatic radical which has 6 to 30 carbon atoms, may contain one or more aromatic rings, may be substituted and may contain aliphatic or cycloaliphatic radicals or alkylaryls or heteroatoms as bridging elements.

It is preferable for Z in formula (1) to be a radical of formula (2):

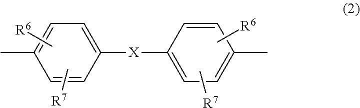

in which

R6 and R7 independently of one another represent H, C1- to C18-alkyl, C1- to C18-alkoxy, halogen such as Cl or Br or in each case optionally substituted aryl or aralkyl, preferably H or Cl- to C12-alkyl, particularly preferably H or C1- to C8-alkyl and very particularly preferably H or methyl, and X represents a single bond, —SO2—, —CO—, —O—, —S—, C1- to C6 alkylene, C₂ to C₅ alkylidene or C₅ to C₆ cycloalkylidene, which may be substituted by C₁ to C₆ alkyl, preferably methyl or ethyl, or else represents C6 to C12 arylene, which may optionally be fused to further aromatic rings containing heteroatoms.

It is preferable when X represents a single bond, C₁ to C₅ alkylene, C₂ to C₅ alkylidene, C₅ to C₆ cycloalkylidene, —O—, —SO—, —CO—, —S—, —SO2— or a radical of formula (2a)

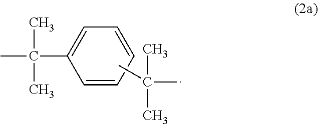

Examples of dihydroxyaryl compounds are: dihydroxybenzenes, dihydroxydiphenyls, bis(hydroxyphenyl)alkanes, bis(hydroxyphenyl)cycloalkanes, bis(hydroxyphenyl)aryls, bis(hydroxyphenyl) ethers, bis(hydroxyphenyl) ketones, bis(hydroxyphenyl) sulfides, bis(hydroxyphenyl) sulfones, bis(hydroxyphenyl) sulfoxides, 1,1′-bis(hydroxyphenyl)diisopropylbenzenes and the ring-alkylated and ring-halogenated compounds thereof.

Dihydroxyaryl compounds suitable for the reaction are, for example, hydroquinone, resorcinol, dihydroxydiphenyl, bis(hydroxyphenyl)alkanes, bis(hydroxyphenyl)cycloalkanes, bis(hydroxyphenyl) sulfides, bis(hydroxyphenyl) ethers, bis(hydroxyphenyl) ketones, bis(hydroxyphenyl) sulfones, bis(hydroxyphenyl) sulfoxides, α,α′-bis(hydroxyphenyl)diisopropylbenzenes and the alkylated, ring-alkylated and ring-halogenated compounds thereof.

Preferred dihydroxyaryl compounds are 4,4′-dihydroxydiphenyl, 2,2-bis(4-hydroxyphenyl)-1-phenylpropane, 1,1-bis(4-hydroxyphenyl)phenylethane, 2,2-bis(4-hydroxyphenyl)propane (bisphenol A (BPA)), 2,4-bis(4-hydroxyphenyl)-2-methylbutane, 1,3-bis [2-(4-hydroxyphenyl)-2-propyl]benzene (bisphenol M), 2,2-bis(3-methyl-4-hydroxyphenyl)propane, bis(3,5-dimethyl-4-hydroxyphenyl)methane, 2,2-bis(3,5-dimethyl-4-hydroxyphenyl)propane, bis(3,5-dimethyl-4-hydroxyphenyl) sulfone, 2,4-bis(3,5-dimethyl-4-hydroxyphenyl)-2-methylbutane, 1,3-bis[2-(3,5-dimethyl-4-hydroxyphenyl)-2-propyl]benzene, 1,1-bis(4-hydroxyphenyl)cyclohexane, and 1,1-bis(4-hydroxyphenyl)-3,3,5-trimethylcyclohexane (bisphenol TMC (BPTMC)), and also the dihydroxyaryl compounds of formulas (III) to (V)

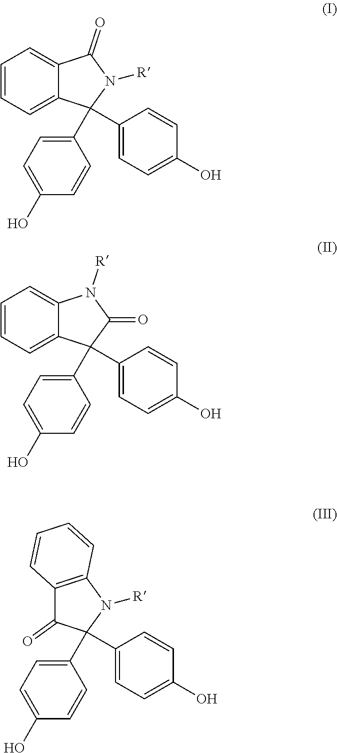

where R′ in each case represents C₁-C₄-alkyl, aralkyl or aryl, preferably methyl or phenyl.

Particularly preferred dihydroxyaryl compounds are 4,4′-dihydroxydiphenyl, 1,1-bis(4-hydroxyphenyl)phenylethane, 2,2-bis(4-hydroxyphenyl)propane (bisphenol A (BPA)), 2,2-bis(3,5-dimethyl-4-hydroxyphenyl)propane, 1,1-bis(4-hydroxyphenyl)cyclohexane and 1,1-bis(4-hydroxyphenyl)-3,3,5-trimethylcyclohexane (bisphenol TMC (BPTMC)), and the dihydroxy compounds of formulas (III), (IV) and (V), where R′ in each case represents C₁-C₄-alkyl, aralkyl or aryl, preferably methyl or phenyl.

These and further suitable dihydroxyaryl compounds are described, for example, in U.S. Pat. Nos. 2,999,835 A, 3,148,172 A, 2,991,273 A, 3,271,367 A, 4,982,014 A and 2,999,846 A, in German published specifications DE 1 570 703 A1, DE 2 063 050 A1, DE 2 036 052 A1, DE 2 211 956 A1 and DE 3 832 396 A1, in French patent FR 1 561 518 A1, in the monograph “H. Schnell, Chemistry and Physics of Polycarbonates, Interscience Publishers, New York 1964, p. 28 ff.; p.102 ff.”, and in “D.G. Legrand, J.T. Bendler, Handbook of Polycarbonate Science and Technology, Marcel Dekker New York 2000, p. 72ff.”.

In the case of the reaction for provision of homopolycarbonate, only one dihydroxyaryl compound is used; in the case of copolycarbonates, two or more different dihydroxyaryl compounds are used. The dihydroxyaryl compound used or the two or more different dihydroxyaryl compounds used, similarly to all other chemicals and auxiliaries added to the synthesis, may be contaminated with the contaminants from their own synthesis, handling and storage. However, it is desirable to work with raw materials of maximum purity.

Any branching agents or branching agent mixtures to be used are added to the synthesis in the same manner. Compounds typically used are trisphenols, quaterphenols or acyl chlorides of tri- or tetracarboxylic acids, or else mixtures of the polyphenols or of the acyl chlorides.

The alkali metal salt used for formation of the alkali metal salt of the dihydroxyaryl compounds may be an alkali metal hydroxide solution comprising hydroxides from the group of: hydroxides of Na, K, Li, preference being given to sodium hydroxide solution, and is used in the novel process preferably as a 10% to 55% by weight solution.

The reaction between phosgene and the alkali metal salt of the dihydroxyaryl compound can be accelerated by catalysts, such as tertiary amines, N-alkylpiperidines or onium salts. Preference is given to using tributylamine, triethylamine and N-ethylpiperidine.

The amine catalyst used may be open-chain or cyclic, particular preference being given to triethylamine and ethylpiperidine. The catalyst is used in the novel process preferably as a 1% to 55% by weight solution.

Onium salts are understood here to mean compounds such as NR4X where R may be an alkyl and/or aryl radical and/or an H and X is an anion.

The polycarbonates may be modified in a deliberate and controlled manner by the use of small amounts of chain terminators and branching agents. Suitable chain terminators and branching agents are known in the literature. Some are described, for example, in DE-A 38 33 953. Chain terminators used with preference are phenol or alkylphenols, especially phenol, p-tert-butylphenol, isooctylphenol, cumylphenol, chlorocarbonic esters thereof or acyl chlorides of monocarboxylic acids, or mixtures of these chain terminators. Preferred chain terminators are phenol, cumylphenol, isooctylphenol, para-tert-butylphenol.

Examples of compounds suitable as branching agents are aromatic or aliphatic compounds having more than three, preferably three or four, hydroxyl groups.

Particularly suitable examples having three, or more than three, phenolic hydroxyl groups are phloroglucinol, 4,6-dimethyl-2,4,6-tri(4-hydroxyphenyl)hept-2-ene, 4,6-dimethyl-2,4,6-tri(4-hydroxyphenyl)heptane, 1,3,5-tri(4-hydroxyphenyl)benzene, 1,1,1-tri(4-hydroxyphenyl)ethane, tri(4-hydroxyphenyl)phenylmethane, 2,2-bis[4,4-bis(4-hydroxyphenyl)cyclohexyl]propane 2,4-bis(4-hydroxyphenylisopropyl)phenol, tetra(4-hydroxyphenyl)methane.

Examples of other trifunctional compounds suitable as branching agents are 2,4-dihydroxybenzoic acid, trimesic acid, cyanuric chloride, and 3,3-bis(3-methyl-4-hydroxyphenyl)-2-oxo-2,3-dihydroindole.

Particularly preferred branching agents are 3,3-bis(3-methyl-4-hydroxyphenyl)-2-oxo-2,3-dihydroindole and 1,1,1-tri(4-hydroxyphenyl)ethane.

For the provision of polycarbonate, it is likewise possible first to produce diphenyl carbonate from the phosgene obtained in accordance with the invention and then to react at least one dihydroxyaryl compound with diphenyl carbonate.

The invention further provides for the incorporation of the abovementioned process into a process sequence for production of carbon monoxide. The process is preferably operated in combination with a process selected from the group of steam reforming, RWGS or CO₂ electrolysis.

The invention further provides for the batchwise performance of the abovementioned process, characterized in that the catalytic cracking of methanol is operated flexibly. This involves storing methanol in an intermediate storage means (for example a tank) and feeding it into the abovementioned process when required. This method is more preferably operated in combination with a further process selected from the group of steam reforming, RWGS or CO₂ electrolysis.

This combination is configured such that all processes can be run flexibly.

The invention further provides for the use of carbon monoxide provided as process product of a catalytic degradation of methanol for production of phosgene.

In respect of this subject matter, the aforementioned preferred embodiments of the features defined for the process for producing the carbon monoxide provided are applicable mutatis mutandis, especially the features specified for the production of methanol and the catalytic degradation of methanol.

The invention further provides a multicomponent system for production of phosgene from chlorine and carbon monoxide, at least comprising
i) as a first component carbon monoxide as process product from a catalytic degradation of methanol and
ii) as a second component an apparatus at least comprising
   at least one feed conduit containing the first component, and
   at least one apparatus for production of phosgene that has at least one inlet for carbon monoxide in fluid connection with said feed conduit, and at least one inlet for chlorine and at least one outlet for phosgene.

A fluid connection is understood to mean an apparatus which connects apparatus components to one another and through which a substance that may be in any state of matter can be transported as a stream of matter from one apparatus component to the next, for example an inlet in the form of a pipe. The expression “are in fluid connection” means that named apparatus components are connected to one another via a fluid connection.

A “reaction space” is a volume of an apparatus in which the co-reactants taking part in a chemical reaction are present together and in which the chemical reaction takes place. For a chemical reaction, this may be, for example, the volume of a vessel or container in which the co-reactants are present together and are to be reacted.

Preferred embodiments of the provision of the first component are already mentioned in the summary of the process of the invention and are applicable mutatis mutandis to the multicomponent system.

In a preferred embodiment of the multicomponent system, the at least one feed conduit containing the first component is additionally in fluid connection with an apparatus for production of the first component, where the apparatus for production of the first component comprises at least one reaction space having at least one inlet for methanol, at least one catalyst and at least one outlet for a product gas comprising the first component, where the outlet for said product gas is in fluid connection with the at least one feed conduit for the second component that contains the first component, optionally via an apparatus for purification. The apparatus for production of the first component preferably additionally comprises at least heating element as an apparatus for supply of thermal energy to the reaction space and/or for evaporation of the methanol. It is particularly preferable in turn when the heating element is in fluid connection with at least one source selected from a source for hydrogen provided from renewable energy as fuel or a source for methane provided from a biological source as fuel, or the heating element is connected to a source for provision of electrical energy from renewable energy for conversion to thermal energy. It is particularly preferable here in turn when the renewable energy used is either wind power, solar energy, hydro power, bio energy or mixtures thereof.

The catalyst present with preference in the apparatus for production of the first component is selected as in the process described above.

It has been found to be advantageous when at least one inlet for methanol in the apparatus for production of the first component is in fluid connection with a methanol source containing methanol that has been obtained from the reaction of the components of a gas stream comprising at least COₓ with x=1 or 2 and preferably additionally hydrogen gas.

For this purpose, the inlet for methanol is in fluid connection with a storage vessel in which the corresponding methanol is stored, or the inlet for methanol is in fluid connection with an apparatus for methanol production. Corresponding apparatuses for methanol production are known to the person skilled in the art from the prior art and can be purchased on the market. These are preferably apparatuses for steam reforming of hydrocarbons (especially of methane), dry reforming of methane, for partial oxidation of hydrocarbons, for gasification of biomass, for electrolysis, for performance of the reverse water-gas shift.

The methanol in the methanol source, in a particularly preferred embodiment, is produced by (i) an apparatus for catalytic conversion of a synthesis gas containing more than 50% by volume of a mixture of carbon dioxide and hydrogen, (ii) by a fermentation apparatus or (iii) by an apparatus for electrochemical conversion of carbon dioxide. Each apparatus (i) to (iii) is supplied here with a COr-containing stream of matter as feed via an inlet. In the case of the fermentation apparatus (ii) and especially of the apparatus for catalytic conversion (i), the feed additionally contains hydrogen.

More preferably, the hydrogen required for steps (i) or (ii) is produced by at least one process selected from the electrolysis of water, partial oxidation or biomass gasification, or mixtures thereof. In this connection, it is particularly preferable when the apparatus for methanol production has at least one inlet which is in fluid connection with the outlet for hydrogen gas from an electrolysis apparatus. It is particularly preferable here when the power supply of the electrolysis apparatus is in contact with a source of electrical power produced from renewable energy. It is particularly preferable here in turn when the renewable energy used is either wind power, solar energy, hydro power, bio energy or mixtures thereof.

In a particularly preferred embodiment, the at least one feed conduit to the multicomponent system that contains the first component is additionally in fluid connection with an apparatus for production of the first component, where the apparatus for production of the first component comprises at least one inlet for methanol, at least one catalyst and at least one outlet for a product gas comprising the first component, where the outlet for said product gas in the apparatus for production of the first component is in fluid connection with the at least one feed conduit for the second component that contains the first component via an apparatus for purification, and the apparatus for purification comprises at least one inlet for said product gas, at least one outlet for methanol separated off, at least one outlet for hydrogen gas separated off, and at least one outlet for the first component, and the outlet for the first component is in fluid connection with the at least one feed conduit for the second component that contains the first component.

In this particularly preferred embodiment of the multicomponent system, a particular preference is given in accordance with the invention to an embodiment in which the outlet for hydrogen gas in the apparatus for purification is in fluid connection with an apparatus for hydrogenation, preferably with an apparatus for hydrogenation of carbon dioxide to methanol as an apparatus for methanol production.

In the particularly preferred embodiment of the multicomponent system comprising a fluid connection via an apparatus for purification, very particular preference is given in accordance with the invention to an embodiment in which the outlet for methanol from the apparatus for purification is in fluid connection with the apparatus for catalytic degradation of methanol.

A further preferred embodiment of the multicomponent system is characterized in that the apparatus for production of the first component comprises at least one apparatus for evaporation of methanol.

A further preferred embodiment of the multicomponent system is characterized in that the apparatus for production of the first component comprises at least one heating element as apparatus for introduction of thermal energy into the reaction space and/or into the apparatus for evaporation of methanol.

The phosgene produced by the multicomponent system is preferably utilized as described above by means of a further step in which the phosgene formed in the apparatus for production of phosgene is fed to a reaction with at least one organic amine or with at least one dihydroxyaryl compound and chemically converted. For that reason, it is preferable when at least one outlet for phosgene in the apparatus for production of phosgene is in fluid connection with at least one apparatus for phosgenation of organic amine or with at least one apparatus for production of polycarbonate. Corresponding apparatuses for phosgenation of aromatic amines or for production of polycarbonate are known to the person skilled in the art from the prior art.

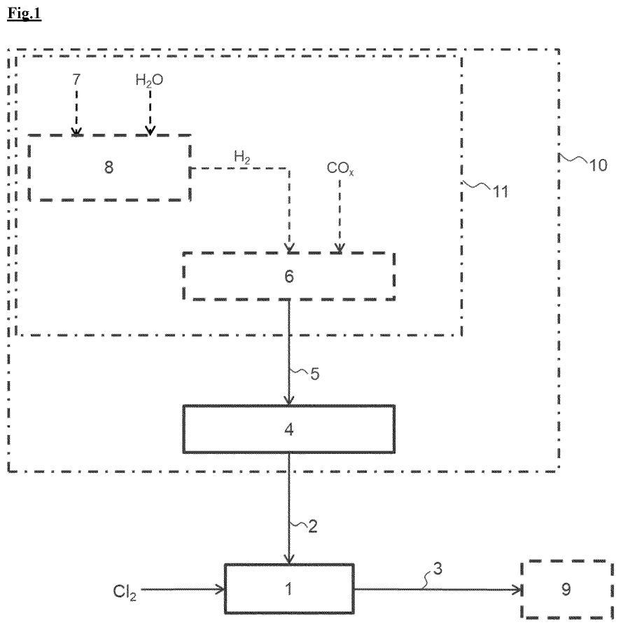

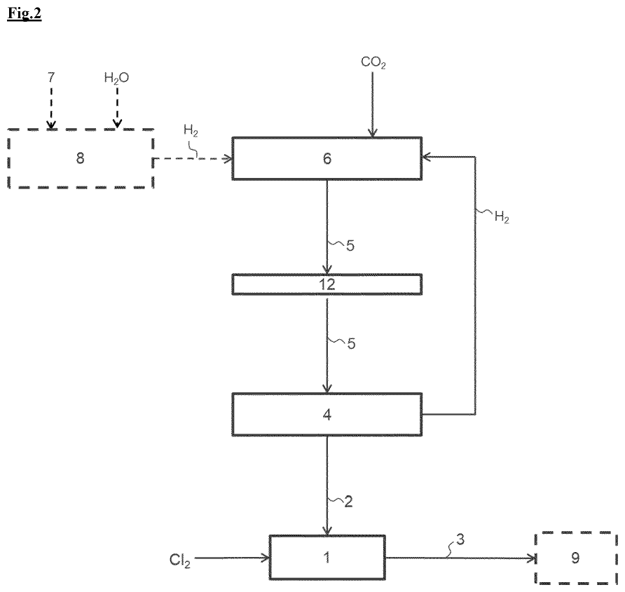

The process and preferred embodiments thereof are illustrated by way of example in the figures FIG. 1 and FIG. 2, without restricting the subject matter of the invention thereto. Arrows in the figure symbolize the flow of substances, energy or heat between process steps/apparatus parts in which the corresponding process steps proceed. Dashed lines in the figures denote parts of preferred embodiments of individual above-described features of the process. Dashed-and-dotted frames illustrate the region of origin of the raw materials provided for the process of the invention, methanol and carbon monoxide here. The following reference numerals are used in the figures:
1 Production of phosgene from chlorine and carbon monoxide
2 Carbon monoxide provided, which is a process product of a catalytic degradation of methanol (4)
3 Phosgene
4 Catalytic degradation of methanol
5 Methanol provided, which is a process product of a reaction 6 selected from an electrochemical reaction, a homogeneously catalyzed reaction or a heterogeneously catalyzed reaction
6 Production of methanol by reaction of a gas stream with COₓ in which x=1 or 2 and with optionally hydrogen (preferably of at least CO₂ and hydrogen), where the methanol produced is a process product of a reaction selected from an electrochemical reaction, a homogeneously catalyzed reaction or a heterogeneously catalyzed reaction
7 Electrical power from renewable energy
8 Electrolysis of water
9 Isocyanate production and/or polycarbonate production
10 Region of origin of the carbon monoxide 2 provided
11 Region of origin of the methanol 5 provided
12 Storage of methanol which is a process product of the production 6
   FIG. 1 shows an embodiment of a process of the invention in which carbon monoxide 2, which is a process product of a catalytic degradation of methanol 4, is provided, for example, by withdrawal of corresponding methanol from a storage tank or from a conduit in fluid connection with the catalytic degradation of methanol 4. This provided carbon monoxide 2 is directed into a production of phosgene 1, where it is reacted with chlorine (Cl₂) to give phosgene 3, and conducted out of the production of phosgene 1, preferably followed by supply of phosgene 3 into an isocyanate production 9. The methanol 5 provided in FIG. 1 is preferably a process product of a production of methanol 6 by reaction of hydrogen with at least CO₂, where the methanol produced is a process product of a reaction selected from an electrochemical reaction, a homogeneously catalyzed reaction or a heterogeneously catalyzed reaction. It is particularly preferable here in turn when hydrogen that has been by electrolysis 8 of water with use of electrical power from renewable energy 7 is used. The region 10 of production of said carbon monoxide 2 provided marks corresponding process steps or plant components that are implemented for the provision by suppliers or the phosgene producer itself. The region 11 of production of the methanol 5 provided marks corresponding process steps or plant components that can be implemented for the provision by a supplier of the carbon monoxide 2 provided, by a further supplier of methanol 5 provided or by the phosgene producer itself.

FIG. 2 shows an embodiment of a process of the invention in which carbon monoxide 2, which is a process product of a catalytic degradation of methanol 4, by withdrawal of corresponding methanol from a storage tank 12 which is in fluid connection with the catalytic degradation of methanol 4, is directed into a production of phosgene 1, where it is reacted with chlorine (C1₂) to give phosgene 3, and conducted out of the production of phosgene 1, preferably followed by supply of phosgene 3 to an isocyanate production 9. The degradation of methanol 4 releases hydrogen gas which is fed to the production of methanol 6. The production of methanol 6 takes place by reaction of hydrogen with at least CO₂, where the methanol produced is a process product of a reaction selected from an electrochemical reaction, a homogeneously catalyzed reaction or a heterogeneously catalyzed reaction. If the amount of hydrogen gas obtained from the degradation of methanol 4 is insufficient for the production of methanol 6, it is particularly preferable when additional hydrogen is utilized, which is produced by electrolysis 8 of water using electrical power from renewable energy 7.

In the context of embodiments of the invention, the following aspects 1 to 22 may be mentioned by way of example:
1. A process for producing phosgene from chlorine and carbon monoxide (1), characterized in that carbon monoxide obtained by catalytic degradation of methanol (4) is provided, and this provided carbon monoxide (2) is reacted with chlorine to give phosgene (3).
2. The process according to aspect 1, characterized in that the catalytic degradation of methanol (4) is preceded by provision of methanol (5) which is the process product of a conversion (6) selected from an electrochemical reaction, a homogeneously catalyzed reaction or a heterogeneously catalyzed reaction.
3. The process according to aspect 1 or 2, characterized in that the catalytic degradation of methanol (4) is preceded by provision of methanol (5) which is the process product of a conversion (6) of a gas stream comprising at least COₓ with x=1 or 2 and optionally hydrogen, preferably CO₂ and hydrogen.
4. The process according to aspect 1 or 2, characterized in that the catalytic degradation of methanol (4) is preceded by provision of methanol which is the process product of a conversion of a carbon monoxide- and hydrogen-containing gas stream which is obtained by a method or a combination of methods from the group of steam reforming of methane, partial oxidation of hydrocarbons, gasification of biomass, electrolysis, reverse water-gas shift.
5. The process according to either of aspects 3 and 4, characterized in that the hydrogen in the hydrogen-containing gas stream is obtained by electrolysis of water (8) using electrical power from renewable energy (7).
6. The process according to any of the preceding aspects, characterized in that, for provision of the methanol (5), it is withdrawn from a storage vessel (12) and/or produced in a continuous process beforehand by at least one process of aspects 2 to 4 and fed in each case to the catalytic degradation (4).
7. The process according to any of aspects 1 to 6, characterized in that, in a further step, the phosgene (3) formed is fed to a reaction (9) with at least one organic amine to obtain a product mixture comprising at least one compound selected from tolylene diisocyanate, methylene diphenyl isocyanate (preferably selected from diphenylmethane 2,2′-diisocyanate, diphenylmethane 2,4′-diisocyanate, diphenylmethane 4,4′-diisocyanate or mixtures thereof), hexamethylene diisocyanate, isophorone diisocyanate, 1,3-bis(isocyanatomethyl)benzene, or cyclohexyl diisocyanate.
8. The process according to any of aspects 1 to 6, characterized in that, in a further step, the phosgene (3) formed is fed to a reaction (9) with at least one dihydroxyaryl compound to obtain a polycarbonate.
9. The process according to any of the preceding aspects, characterized in that, for catalytic degradation of methanol (4), the methanol (5) is broken down catalytically at a temperature of below 500° C., especially of below 400° C.
10. The process according to any of the preceding aspects, characterized in that, for catalytic degradation of methanol (4), the methanol (5) is broken down catalytically at an absolute pressure of below 50 bar, especially below 40 bar.
11. The process according to any of the preceding aspects, characterized in that methanol (5) provided for catalytic degradation (4) is contacted with a catalyst as gaseous methanol and broken down catalytically to form carbon monoxide (2) and optionally additionally hydrogen gas.
12. The process according to any of the preceding aspects, characterized in that, for catalytic degradation of methanol (4), gaseous methanol is passed over a catalyst, the active component of which contains at least one transition metal species.
13. The process according to any of the preceding aspects, characterized in that, for catalytic degradation of methanol (5), a catalyst containing at least one transition metal species applied to a support is used, especially at least one transition metal species selected from group VII, VIII or XI of the Periodic Table of the Elements, applied to a support.
14. The process according to any of preceding aspects, characterized in that
   (i) methanol (5) provided which is fed to the catalytic degradation of methanol (4) is the process product of a reaction of CO₂ with hydrogen gas, and
   (ii) the catalytic degradation (4) of the methanol (5) already provided affords not only carbon monoxide (2) but additionally hydrogen gas, with the proviso that hydrogen gas formed in the catalytic degradation (4) is fed to the reaction of CO₂ with hydrogen gas to give methanol (5).
15. A multicomponent system for production of phosgene from chlorine and carbon monoxide, at least comprising
   i) as a first component carbon monoxide obtained as process product from a catalytic degradation of methanol and
   ii) as a second component an apparatus at least comprising
      at least one feed conduit containing the first component, and
      at least one apparatus for production of phosgene that has at least one inlet for carbon monoxide in fluid connection with said feed conduit, and at least one inlet for chlorine and at least one outlet for phosgene.
16. The multicomponent system according to aspect 15, characterized in that the at least one feed conduit containing the first component is additionally in fluid connection with an apparatus for production of the first component, where the apparatus for production of the first component comprises at least one inlet for methanol, at least one catalyst and at least one outlet for a product gas comprising the first component, where the outlet for said product gas is in fluid connection, optionally via an apparatus for purification, with the at least one feed conduit for the second component that contains the first component.
17. The multicomponent system according to aspect 16, characterized in that the outlet for the product gas in the apparatus for production of the first component is in fluid connection with the at least one feed conduit for the second component that contains the first component via an apparatus for purification, and the apparatus for purification comprises at least one inlet for said product gas, at least one outlet for methanol separated off, at least one outlet for hydrogen gas separated off, and at least one outlet for the first component, and the outlet for the first component is in fluid connection with the at least one feed conduit for the second component that contains the first component.
18. The multicomponent system according to aspect 17, characterized in that the outlet for hydrogen gas in the apparatus for purification is in fluid connection with an apparatus for hydrogenation, preferably with an apparatus for hydrogenation of carbon dioxide to methanol as an apparatus for methanol production.
19. The multicomponent system according to any of aspects 16 to 18, characterized in that the at least one inlet for methanol in the apparatus for production of the first component is in fluid connection with a methanol source containing methanol that has been obtained from the reaction of the components of a gas stream comprising at least COₓ with x=1 or 2 and preferably additionally hydrogen gas.
20. The multicomponent system according to any of aspects 15 to 19, characterized in that the catalyst in the apparatus for production of the first component is selected from at least one transition metal species selected from group VII, VIII or XI of the Periodic Table of the Elements, preferably applied to a support.
21. The multicomponent system according to any of aspects 15 to 20, characterized in that the apparatus for production of the first component comprises at least one heating element as apparatus for introduction of thermal energy into the reaction space and/or into an apparatus for evaporation of methanol.
22. The multicomponent system according to any of aspects 15 to 21, characterized in that at least one outlet for phosgene in the apparatus for production of phosgene is in fluid connection with at least one apparatus for phosgenation of organic amine or with at least one apparatus for production of polycarbonate.
23. The use of carbon monoxide provided as process product of a catalytic degradation of methanol for production of phosgene.

1. A process for producing phosgene from chlorine and carbon monoxide,
the process comprising providing carbon monoxide by a catalytic degradation of methanol, and reacting the carbon monoxide with chlorine to give phosgene.

2. The process as claimed in claim 1, wherein the catalytic degradation of methanol is preceded by a provision of methanol, wherein the methanol is a process product of a conversion selected from an electrochemical reaction, a homogeneously catalyzed reaction or a heterogeneously catalyzed reaction.

3. The process as claimed in claim 1, wherein the catalytic degradation of methanol is preceded by a provision of methanol, wherein the methanol is a process product of a conversion of a gas stream comprising COₓ with x=1 or 2 and optionally hydrogen.

4. The process as claimed in claim 1, wherein the catalytic degradation of methanol is preceded by a provision of methanol, wherein the methanol is a process product of a conversion of a gas stream comprising carbon monoxide and hydrogen, wherein the gas stream is obtained by a method comprising steam reforming of methane, partial oxidation of hydrocarbons, gasification of biomass, electrolysis, reverse water-gas shift, or any combination thereof.

5. The process as claimed in claim 2, wherein the provision of the methanol is withdrawn from a storage vessel and/or produced in a continuous process and fed in each case to the catalytic degradation.

6. The process as claimed in claim 1, wherein the phosgene is fed to a reaction with at least one organic amine to obtain at least one compound selected from tolylene diisocyanate, methylene diphenyl isocyanate hexamethylene diisocyanate, isophorone diisocyanate, 1,3-bis(isocyanatomethyl)benzene, or cyclohexyl diisocyanate.

7. The process as claimed in claim 1, wherein the phosgene is fed to a reaction with at least one dihydroxyaryl compound to obtain a polycarbonate.

8. The process as claimed in claim 1, wherein, for catalytic degradation of methanol, the methanol is broken down catalytically at a temperature below 500° C.

9. The process as claimed in claim 1, wherein, for catalytic degradation of methanol, the methanol is broken down catalytically at an absolute pressure below 50 bar.

10. The process as claimed in claim 1, wherein methanol provided for catalytic degradation is contacted with a catalyst as gaseous methanol and broken down catalytically to form carbon monoxide.

11. The process as claimed in claim 1, wherein, for catalytic degradation of methanol, gaseous methanol is passed over a catalyst, wherein the catalyst comprises an active component which contains at least one transition metal species.

12. The process as claimed in claim 1, wherein, for catalytic degradation of methanol, a catalyst containing at least one transition metal species applied to a support is used.

13. The process as claimed in claim 1, wherein:
(i) methanol provided which is fed to the catalytic degradation of methanol is a process product of a reaction of CO₂ with hydrogen gas, and
(ii) the catalytic degradation of the methanol forms carbon monoxide and hydrogen gas,
wherein the hydrogen gas formed in the catalytic degradation is fed to the reaction of CO₂ with hydrogen gas.

14. A multicomponent system for production of phosgene from chlorine and carbon monoxide, the multicomponent system comprising:
i) as a first component carbon monoxide obtained as process product from a catalytic degradation of methanol, and
ii) as a second component an apparatus at least comprising:
at least one feed conduit containing the first component, and
at least one apparatus for production of phosgene, the apparatus comprising at least one inlet for carbon monoxide in fluid connection with said feed conduit, at least one inlet for chlorine and at least one outlet for phosgene.

15. The multicomponent system as claimed in claim 14, wherein the at least one feed conduit containing the first component is additionally in fluid connection with an apparatus for production of the first component, wherein the apparatus for production of the first component comprises at least one inlet for methanol, at least one catalyst and at least one outlet for a product gas comprising the first component, wherein the outlet for said product gas is in fluid connection, optionally via an apparatus for purification, with the at least one feed conduit of the second component.

16. The multicomponent system as claimed in claim 15, wherein the catalyst in the apparatus for production of the first component is selected from at least one transition metal species selected from group VII, VIII or XI of the Periodic Table of the Elements.

17. The process as claimed in claim 3, wherein the gas stream comprises CO₂ and hydrogen.

18. The process as claimed in claim 6, wherein the methylene diphenyl isocyanate is diphenylmethane 2,2′-diisocyanate, diphenylmethane 2,4′-diisocyanate, diphenylmethane 4,4′-diisocyanate, or any combination thereof.

19. The process as claimed in claim 8, wherein the methanol is broken down catalytically at a temperature below 400° C.

20. The process as claimed in claim 9, wherein the methanol is broken down catalytically at an absolute pressure below 40 bar.
